(19) United States
(12) Patent Application Publication
(10) Pub. No.: US 2012/0214204 A1
(43) Pub. Date: Aug. 23, 2012

(54) CELL CULTURE MEDIA FOR UVC EXPOSURE AND METHODS RELATED THERETO

(75) Inventors: Roger HART, Loveland, CO (US); Robert Michael Boychyn, Basel (CH)

(73) Assignee: AMGEN INC., Thousand Oaks, CA (US)

(21) Appl. No.: 13/399,599

(22) Filed: Feb. 17, 2012

Related U.S. Application Data
(60) Provisional application No. 61/445,988, filed on Feb. 23, 2011.

(51) Int. Cl. C12P 21/00; C12N 5/07; C12N 5/071

(52) U.S. Cl. 435/70.3; 435/404; 435/348

(57) ABSTRACT

The invention relates to cell culture media optimized for exposure to ultraviolet C (UVC) light exposure and related methods.

### CROSS-REFERENCE TO RELATED APPLICATIONS

This application claims the benefit of U.S. Provisional Application No. 61/445,988, filed Feb. 23, 2011, which is incorporated by reference herein.

### FIELD OF THE INVENTION

The invention relates to cell culture media optimized for exposure to ultraviolet C (UVC) light exposure and methods related thereto.

### BACKGROUND OF THE INVENTION

Sterilization of cell culture media that will be used in the manufacture of pharmaceutical products is an important step as part of a process to produce high quality pharmaceutical products to prevent bioburden. This is typically achieved by sterilizing grade filtration (0.2 or 0.1 micron absolute rated filters). Mycoplasma and viral contamination of cellular media and supernatants also poses a large challenge to biopharmaceutical manufacturers worldwide. Several methods have been employed to inactivate and/or remove large or small, enveloped or non-enveloped (or “naked”) DNA or RNA viral particles from solutions. Examples of these approaches include filtration (nano, viral or 0.1 micron), chromatography, batch heat treatment, flow-through High Temperature Short Term (HTST), gamma irradiation, low pH and chemical inactivation (solvents, detergents), and batch or flow-through ultraviolet light C (UVC). HTST is a proven method for the control of viruses, however, it has adverse effects on cell culture media that contain proteinaceous components such as serum. Additionally, chemical treatments to inactivate viral particles have been used, although the frequently toxic nature of these chemicals limits their use in pharmaceutical manufacturing. Moreover, HTST requires dedicated and integrated infrastructure in a plant, which can be a consideration when contemplating the manufacture of pharmaceutical and therapeutic agents.

In addition to the above techniques, UVC technology has been used to treat large-scale protein preparations prior to the purification of these proteins from cellular supernatants. See, for example, U.S. Patent Appl. Publ. No. 20100203610A1. UVC technology relies on the property of light in the ultraviolet wavelength range of the spectrum to disrupt the DNA/RNA of the unwanted organism. The intensity of the UVC treatment, considered to be the UVC dose, is dictated by the intensity of the light flux and the time the liquid is exposed to the UVC light source. The UVC dose provided needs to be sufficient for effective inactivation of the desired organisms, but must not be too high as to disrupt the components of the solution necessary for a robust process including target protein production and quality. Although UVC treatment of media is an effective means for viral inactivation, the present inventors have found that cells grown with media that has been exposed to UVC light prior to growth produce protein titers that are reduced as compared to media that has not been exposed to UVC light.

Thus, there is a need in the art for a UVC treatable cell culture media, and methods for treating cell culture media with UVC for use in pharmaceutical manufacturing. Such methods can be particularly useful for protecting valuable cell lines from viral contamination, saving costs lost as a result of contaminated and unusable media, and increasing the efficiency of protein production by such cell lines. Accordingly, the development of such methods can have wide application in the manufacture of biopharmaceuticals.

### SUMMARY OF THE INVENTION

Provided herein is a cell culture media comprising (a) a base media that is exposed to UVC light; and (b) an additive package comprising UV sensitive media components that is added to said base media after UVC exposure. In one embodiment, the base media does not comprise at least one, two, three, four, five, six, seven, eight, nine, ten, eleven, twelve, or thirteen components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In another embodiment the additive package comprises at least one, two, three, four, five, six, seven, eight, nine, ten, eleven, twelve, or thirteen components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In a further embodiment, the base media is in a powder or liquid form, and the additive package is in a powder or liquid form. In yet another embodiment the media is suitable for culture of mammalian cells, while in still another embodiment the media is suitable for culture of insect cells.

Also provided herein is a method for making a UVC exposed cell culture media formulation, said method comprising the steps of (a) exposing a base media to UVC light; and (b) adding an additive package comprising UV sensitive components to said UVC exposed base media. In one embodiment the base media does not comprise at least one, two, three, four, five, six, seven, eight, nine, ten, eleven, twelve, or thirteen components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In a further embodiment the additive package comprises at least one, two, three, four, five, six, seven, eight, nine, ten, eleven, twelve, or thirteen components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In another embodiment the UVC light is at a wavelength of about 254 nm. In a further embodiment, the base media is exposed to UVC light at an energy density of about 25 to about 350 mJ/cm². In yet another embodiment, the base media is exposed to UVC light at an energy density of about 125 mJ/cm², while in a further embodiment the base media is exposed to UVC light at an energy density of about 175 mJ/cm². In still another embodiment, the step of exposing the base media to UVC light is sufficient to damage the nucleic acids of any non-enveloped viruses in the base media. In another embodiment the UVC light is delivered using a thin film UVC reactor, while in a further embodiment the UVC light is delivered using a helical UVC reactor.

Also provided herein is a method for producing a protein, said method comprising the steps of (a) exposing a base media to UVC light; (b) adding an additive package comprising UV sensitive media components to said UVC exposed base media; and (c) culturing cells in the UVC treated media such that a desired protein is produced. In one embodiment the base media does not comprise at least one, two, three, four, five, six, seven, eight, nine, ten, eleven, twelve, or thirteen components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In a further embodiment the additive package comprises at least one, two, three, four, five, six, seven, eight, nine, ten, eleven, twelve, or thirteen components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In another embodiment the UVC light is at a wavelength of about 254 nm. In a further embodiment, the base media is exposed to UVC light at an energy density of about 25 to about 350 mJ/cm². In yet another embodiment, the base media is exposed to UVC light at an energy density of about 125 mJ/cm², while in a further embodiment the base media is exposed to UVC light at an energy density of about 175 mJ/cm². In still another embodiment, the step of exposing the base media to UVC light is sufficient to damage the nucleic acids of any non-enveloped viruses in the base media. In another embodiment the UVC light is delivered using a thin film UVC reactor, while in a further embodiment the UVC light is delivered using a helical UVC reactor. In one embodiment, the cells are CHO cells. In a further embodiment the protein is recombinant human erythropoietin.

### BRIEF DESCRIPTION OF THE DRAWINGS

FIG. 1 describes the HPLC titer results from Example 1. Shown are results for the control untreated media and the various UVC treated media groups. The Y-axis represents mg/L of recombinant human erythropoietin and the X-axis represents the 3 different harvest cycles and also the total yield.

FIG. 2 describes the Bradford assay results from Example 1. Shown are results for the control untreated media and the various UVC treated media groups. The Y-axis represents mg/L of recombinant protein and the X-axis represents the 3 different harvest cycles and also the total yield.

FIG. 3 describes the HPLC titer results from Example 2. Shown are results for the control untreated media and the various UVC treated media groups. The Y-axis represents mg/L of recombinant human erythropoietin and the X-axis represents the 3 different harvests.

FIG. 4 describes the Bradford assay results from Example 2. Shown are results for the control untreated media and the various UVC treated media groups. The Y-axis represents mg/L of recombinant protein and the X-axis represents the 3 different harvest cycles.

FIG. 5 describes the HPLC titer results from Example 3. Shown are results for the control untreated media and the various UVC treated media groups. The Y-axis represents mg/RB of recombinant human erythropoietin and the X-axis represents the 3 different harvest cycles and the sum of the harvest cycles.

### DETAILED DESCRIPTION OF THE INVENTION

The section headings utilized herein are for organizational purposes only and are not to be construed as limiting the subject matter described therein. All references cited in this application are expressly incorporated by reference herein. Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular.

The present invention addresses the need in the art for a UVC treatable cell culture media. Several obstacles had to be overcome in developing the novel formulations and methods of the present invention. Starting with the original media, the formulation of a stable, new base media was attempted by removing the UV sensitive components from the original media. Quite unexpectedly, however, it was found that removing the UV sensitive components from the original media led to certain instability and solubility issues in both the new base media that does not comprise UV sensitive components, and the additive package comprising UV sensitive components.

The inability to predict the behavior of the separated mixtures derives from chemical interactions among and between the numerous constituents within the mixtures. More specifically, the solubility problem with the additive package is thought to be derived from an interaction of protons in the self buffering systems. Further, an instability problem with the new base media and additive package could have also been derived from other interactions, such as through reactive oxygen species which can be produced by exposure to UVC radiation.

Unexpectedly, it was found that that the additive package was not stable. The additive package was not originally soluble or thermally stable when it was removed from the original base media. In order to regain the stability similar to the original media, it was necessary to adjust the pH of the mixture by adding titrant to make it soluble and stable.

A further unexpected observance was related to an interaction in the new base media that does not comprise UV sensitive components when treated with UV, resulting in damage to key components of the base media. This damage would not have been predictable because components in the additive package ordinarily quench the reactive species in the original media, thereby protecting it from UV. Nonlimiting examples of potential quenchers not present in the new base media because they were placed in the additive package include pyridoxal and pyridoxal. Nonlimiting examples of quenchers remaining in the new base media which could have been damaged due to the absence of additional quenchers include pyruvate. Nonlimiting examples of key components remaining in the new base media which could have been damaged due to the absence of additional quenchers include fetal calf serum proteins.

Surprisingly, however, the additive package is self-stabilizing in certain aspects like a complex mixture (e.g., the original base media). The natural pH of the additive package is approximately 6.7, which is approximately the same as the base media.

An additional unexpected effect is related to the type of UVC reactor and related dose distribution. Several process UVC reactors, e.g., laminar or thin-film reactors, produce a wide dose distribution, typically with a high dose tail. The new media receives this wide distribution (and overexposure), but it is surprisingly not damaged significantly as evidenced by the titer remediation. Previous studies used a helical UVC reactor which produces a tight dose distribution as compared to a thin-film reactor. These studies showed that UVC treatment results in decreased titer (product concentration). When the original media was thin-film UVC treated, the result was total process failure, i.e., a complete lack of attachment of cells to roller bottles and a lack of cell growth. Accordingly, in one aspect the invention provides a cell culture media suitable for UVC exposure, wherein said media comprises a base media and an additive package comprising UV sensitive media components that is added to the base media after UVC exposure. In a further aspect, the invention further provides a method for making a UVC exposed cell culture media formulation, said method comprising the steps of formulating a base media, exposing the base media to UVC light, and adding an additive package comprising UV sensitive components to the UVC exposed base media. In another aspect the invention further provides a method for producing a protein, said method comprising the steps of exposing a base media to UVC light, adding an additive package comprising UV sensitive media components to the UVC exposed base media, and culturing cells in the UVC treated media such that a desired protein is produced.

### UV-Sensitive Media Components

As will be discussed further herein below, cell culture media comprises many different components that support cell proliferation in an in vitro setting. It has been found that certain media components are sensitive to UVC light and degrade when exposed to UVC light. This in turn leads to reduced protein production from the cell culture. To overcome this deleterious impact to the protein product titer observed with UVC treatment of cell culture media, a new base media that is suitable for UVC exposure is required. Accordingly, an object of the invention is to provide a base media that does not comprise the UV sensitive components, and an additive package that comprises UV sensitive components. As used herein, “base media” refers to a liquid, powdered, or other form of cell culture media that does not comprise certain UV sensitive components. As used herein, “additive package” refers to a liquid, powdered, or other form of UV sensitive component(s) to be added to the base media after the base media has been exposed to UVC light.

In one embodiment, the base media is in a powdered form and the additive package is in a powdered form. In another embodiment, the base media is in a powdered form and the additive package is in a liquid form. In yet another embodiment, the base media is in a liquid form and the additive package is in a powdered form. In another embodiment, the base media is in a liquid form and the additive package is in a liquid form.

In one embodiment, the base media suitable for UVC exposure is a liquid media that does not comprise at least one component selected from the group consisting of lipoic acid, histidine, phenylalanine, tryptophan, tyrosine, folic acid, niacinamide, pyridoxal, pyridoxine, riboflavin, thiamine, methotrexate, and vitamin B12 (cyanacobalamin). In another embodiment, the base media suitable for UVC exposure is a liquid media that does not comprise at least two components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In one embodiment, the base media suitable for UVC exposure is a liquid media that does not comprise at least three components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In a further embodiment, the base media suitable for UVC exposure is a liquid media that does not comprise at least four components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In yet a further embodiment, the base media suitable for UVC exposure is a liquid media that does not comprise at least five components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In still a further embodiment, the base media suitable for UVC exposure is a liquid media that does not comprise at least six components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In one embodiment, the base media suitable for UVC exposure is a liquid media that does not comprise at least seven components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In yet another embodiment, the base media suitable for UVC exposure is a liquid media that does not comprise at least eight components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In a further embodiment, the base media suitable for UVC exposure is a liquid media that does not comprise at least nine components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In still a further embodiment, the base media suitable for UVC exposure is a liquid media that does not comprise at least ten components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In yet another embodiment, the base media suitable for UVC exposure is a liquid media that does not comprise at least eleven components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In one embodiment, the base media suitable for UVC exposure is a liquid media that does not comprise at least twelve components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In still another embodiment, the base media suitable for UVC exposure is a liquid media that does not comprise lipoic acid, histidine, phenylalanine, tryptophan, tyrosine, folic acid, niacinamide, pyridoxal, pyridoxine, riboflavin, thiamine, methotrexate, and vitamin B12.

In one embodiment, the base media suitable for UVC exposure is a powdered media that does not comprise at least one component selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In another embodiment, the base media suitable for UVC exposure is a powdered media that does not comprise at least two components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In yet another embodiment, the base media suitable for UVC exposure is a powdered media that does not comprise at least three components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In still another embodiment, the base media suitable for UVC exposure is a powdered media that does not comprise at least four components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In a further embodiment, the base media suitable for UVC exposure is a powdered media that does not comprise at least five components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In yet a further embodiment, the base media suitable for UVC exposure is a powdered media that does not comprise at least six components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In still a further embodiment, the base media suitable for UVC exposure is a powdered media that does not comprise at least seven components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In yet another embodiment, the base media suitable for UVC exposure is a powdered media that does not comprise at least eight components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In still another embodiment, the base media suitable for UVC exposure is a powdered media that does not comprise at least nine components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In another embodiment, the base media suitable for UVC exposure is a powdered media that does not comprise at least ten components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In a further embodiment, the base media suitable for UVC exposure is a powdered media that does not comprise at least eleven components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In yet a further embodiment, the base media suitable for UVC exposure is a powdered media that does not comprise at least twelve components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In still a further embodiment, the base media suitable for UVC exposure is a powdered media that does not comprise lipoic acid, histidine, phenylalanine, tryptophan, tyrosine, folic acid, niacinamide, pyridoxal, pyridoxine, riboflavin, thiamine, methotrexate, and vitamin B12.

In one embodiment, the additive package is a liquid that comprises at least one component selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In another embodiment, the additive package is a liquid that comprises at least two components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In still another embodiment, the additive package is a liquid that comprises at least three components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In yet another embodiment, the additive package is a liquid that comprises at least four components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In still a further embodiment, the additive package is a liquid that comprises at least five components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In a further embodiment, the additive package is a liquid that comprises at least six components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In still a further embodiment, the additive package is a liquid that comprises at least seven components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In one embodiment, the additive package is a liquid that comprises at least eight components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In another embodiment, the additive package is a liquid that comprises at least nine components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In yet another embodiment, the additive package is a liquid that comprises at least ten components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In still another embodiment, the additive package is a liquid that comprises at least eleven components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In yet a further embodiment, the additive package is a liquid that comprises at least twelve components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In another embodiment, the additive package is a liquid that comprises lipoic acid, histidine, phenylalanine, tryptophan, tyrosine, folic acid, niacinamide, pyridoxal, pyridoxine, riboflavin, thiamine, methotrexate, and vitamin B12.

In one embodiment, the additive package is powdered and comprises at least one component selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In still another embodiment, the additive package is powdered and comprises at least two components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In one embodiment, the additive package is powdered and comprises at least three components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In still a further embodiment, the additive package is powdered and comprises at least four components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In yet another embodiment, the additive package is powdered and comprises at least five components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In still another embodiment, the additive package is powdered and comprises at least six components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In yet another embodiment, the additive package is powdered and comprises at least seven components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In a further embodiment, the additive package is powdered and comprises at least eight components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In yet another embodiment, the additive package is powdered and comprises at least nine components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In still a further embodiment, the additive package is powdered and comprises at least ten components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In yet another embodiment, the additive package is powdered and comprises at least eleven components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In still another embodiment, the additive package is powdered and comprises at least twelve components selected from the group consisting of lipoic acid, histidine, phenylalanine, tryptophan, tyrosine, folic acid, niacinamide, pyridoxal, pyridoxine, riboflavin, thiamine, methotrexate, and vitamin B12. In another embodiment, the additive package is powdered and comprises lipoic acid, histidine, phenylalanine, tryptophan, tyrosine, folic acid, niacinamide, pyridoxal, pyridoxine, riboflavin, thiamine, methotrexate, and vitamin B12.

In a further embodiment, the invention relates to a kit comprising a base media suitable for UVC exposure and an additive package.

In one embodiment, the base media that has been exposed to UVC light further comprises at least one component selected from the group consisting of lipoic acid, histidine, phenylalanine, tryptophan, tyrosine, folic acid, niacinamide, pyridoxal, pyridoxine, riboflavin, thiamine, methotrexate, and vitamin B12, which is added to the UVC, exposed base media. In another embodiment, the base media that has been exposed to UVC light further comprises at least two components selected from the group consisting of lipoic acid, histidine, phenylalanine, tryptophan, tyrosine, folic acid, niacinamide, pyridoxal, pyridoxine, riboflavin, thiamine, methotrexate, and vitamin B12, which are added to the UVC exposed base media. In yet another embodiment, the base media that has been exposed to UVC light further comprises at least four components selected from the group consisting of lipoic acid, histidine, phenylalanine, tryptophan, tyrosine, folic acid, niacinamide, pyridoxal, pyridoxine, riboflavin, thiamine, methotrexate, and vitamin B12, which are added to the UVC exposed base media. In still another embodiment, the base media that has been exposed to UVC light further comprises at least five components selected from the group consisting of lipoic acid, histidine, phenylalanine, tryptophan, tyrosine, folic acid, niacinamide, pyridoxal, pyridoxine, riboflavin, thiamine, methotrexate, and vitamin B12, which are added to the UVC exposed base media. In still another embodiment, the base media that has been exposed to UVC light further comprises at least six components selected from the group consisting of lipoic acid, histidine, phenylalanine, tryptophan, tyrosine, folic acid, niacinamide, pyridoxal, pyridoxine, riboflavin, thiamine, methotrexate, and vitamin B12, which are added to the UVC exposed base media. In another embodiment, the base media that has been exposed to UVC light further comprises at least seven components selected from the group consisting of lipoic acid, histidine, phenylalanine, tryptophan, tyrosine, folic acid, niacinamide, pyridoxal, pyridoxine, riboflavin, thiamine, methotrexate, and vitamin B12, which are added to the UVC exposed base media. In yet another embodiment, the base media that has been exposed to UVC light further comprises at least eight components selected from the group consisting of lipoic acid, histidine, phenylalanine, tryptophan, tyrosine, folic acid, niacinamide, pyridoxal, pyridoxine, riboflavin, thiamine, methotrexate, and vitamin B12, which are added to the UVC exposed base media. In yet another embodiment, the base media that has been exposed to UVC light further comprises at least nine components selected from the group consisting of lipoic acid, histidine, phenylalanine, tryptophan, tyrosine, folic acid, niacinamide, pyridoxal, pyridoxine, riboflavin, thiamine, methotrexate, and vitamin B12, which are added to the UVC exposed base media. In still another embodiment, the base media that has been exposed to UVC light further comprises at least ten components selected from the group consisting of lipoic acid, histidine, phenylalanine, tryptophan, tyrosine, folic acid, niacinamide, pyridoxal, pyridoxine, riboflavin, thiamine, methotrexate, and vitamin B12, which are added to the UVC exposed base media. In still another embodiment, the base media that has been exposed to UVC light further comprises at least eleven components selected from the group consisting of lipoic acid, histidine, phenylalanine, tryptophan, tyrosine, folic acid, niacinamide, pyridoxal, pyridoxine, riboflavin, thiamine, methotrexate, and vitamin B12, which are added to the UVC exposed base media. In yet another embodiment, the base media that has been exposed to UVC light further comprises at least twelve components selected from the group consisting of lipoic acid, histidine, phenylalanine, tryptophan, tyrosine, folic acid, niacinamide, pyridoxal, pyridoxine, riboflavin, thiamine, methotrexate, and vitamin B12, which are added to the UVC exposed base media. In another embodiment, the base media that has been exposed to UVC light further comprises lipoic acid, histidine, phenylalanine, tryptophan, tyrosine, folic acid, niacinamide, pyridoxal, pyridoxine, riboflavin, thiamine, methotrexate, and vitamin B12, which are added to the UVC exposed base media.

In the following descriptions of concentrations for the UV sensitive components, the specific concentrations and ranges of concentrations are the final concentrations for the 1× media. The skilled practitioner would readily envision the necessary concentrations for a 2× media solution (i.e., double the given concentrations and ranges), a 3× media solution (i.e., triple the given concentrations), and the like.

In one embodiment, the base media that has been exposed to UVC light further comprises folic acid, which is added to the UVC exposed base media to yield a concentration of about 0.1 to about 10.0 mg/L. In another embodiment, the base media that has been exposed to UVC light further comprises folic acid, which is added to the UVC exposed base media to yield a concentration of about 0.5 to about 5.0 mg/L. In still another embodiment, the base media that has been exposed to UVC light further comprises folic acid, which is added to the UVC exposed base media to yield a concentration of about 1.0 to about 3.0 mg/L. In yet another embodiment, the base media that has been exposed to UVC light further comprises folic acid, which is added to the UVC exposed base media to yield a concentration of about 2.0 to about 3.0 mg/L. In still another embodiment, the base media that has been exposed to UVC light further comprises folic acid, which is added to the UVC exposed base media to yield a concentration of about 2.0 mg/L. In a further embodiment, the base media that has been exposed to UVC light further comprises folic acid, which is added to the UVC exposed base media to yield a concentration of about 2.5 mg/L. In yet another embodiment, the base media that has been exposed to UVC light further comprises folic acid, which is added to the UVC exposed base media to yield a concentration of about 3.0 mg/L.

In one embodiment, the base media that has been exposed to UVC light further comprises histidine, which is added to the UVC exposed base media to yield a concentration to yield a concentration of about 1.0 to about 100.0 mg/L. In another embodiment, the base media that has been exposed to UVC light further comprises histidine, which is added to the UVC exposed base media to yield a concentration of about 10.0 to about 90.0 mg/L. In another embodiment, the base media that has been exposed to UVC light further comprises histidine, which is added to the UVC exposed base media to yield a concentration of about 10.0 to about 80.0 mg/L. In still another embodiment, the base media that has been exposed to UVC light further comprises histidine, which is added to the UVC exposed base media to yield a concentration of about 10.0 to about 70.0 mg/L. In yet another embodiment, the base media that has been exposed to UVC light further comprises histidine, which is added to the UVC exposed base media to yield a concentration of about 15.0 to about 55.0 mg/L. In yet another embodiment, the base media that has been exposed to UVC light further comprises histidine, which is added to the UVC exposed base media to yield a concentration of about 20.0 to about 50.0 mg/L. In still another embodiment, the base media that has been exposed to UVC light further comprises histidine, which is added to the UVC exposed base media to yield a concentration of about 25.0 to about 40.0 mg/L. In yet another embodiment, the base media that has been exposed to UVC light further comprises histidine, which is added to the UVC exposed base media to yield a concentration of about 25.0 to about 35.0 mg/L. In another embodiment, the base media that has been exposed to UVC light further comprises histidine, which is added to the UVC exposed base media to yield a concentration of about 30.0 to about 35.0 mg/L. In yet another embodiment, the base media that has been exposed to UVC light further comprises histidine, which is added to the UVC exposed base media to yield a concentration of about 30.0 to about 33.0 mg/L. In still another embodiment, the base media that has been exposed to UVC light further comprises histidine, which is added to the UVC exposed base media to yield a concentration of about 31.0 to about 32.0 mg/L. In yet another embodiment, the base media that has been exposed to UVC light further comprises histidine, which is added to the UVC exposed base media to yield a concentration of about 31.0 mg/L. In still another embodiment, the base media that has been exposed to UVC light further comprises histidine, which is added to the UVC exposed base media to yield a concentration of about 32.0 mg/L. In another embodiment, the base media that has been exposed to UVC light further comprises histidine, which is added to the UVC exposed base media to yield a concentration of about 31.5 mg/L.

In one embodiment, the base media that has been exposed to UVC light further comprises phenylalanine, which is added to the UVC exposed base media to yield a concentration to yield a concentration of about 1.0 to about 100.0 mg/L. In another embodiment, the base media that has been exposed to UVC light further comprises phenylalanine, which is added to the UVC exposed base media to yield a concentration of about 10.0 to about 90.0 mg/L. In yet another embodiment, the base media that has been exposed to UVC light further comprises phenylalanine, which is added to the UVC exposed base media to yield a concentration of about 10.0 to about 80.0 mg/L. In another embodiment, the base media that has been exposed to UVC light further comprises phenylalanine, which is added to the UVC exposed base media to yield a concentration of about 10.0 to about 70.0 mg/L. In still another embodiment, the base media that has been exposed to UVC light further comprises phenylalanine, which is added to the UVC exposed base media to yield a concentration of about 15.0 to about 55.0 mg/L. In another embodiment, the base media that has been exposed to UVC light further comprises phenylalanine, which is added to the UVC exposed base media to yield a concentration of about 20.0 to about 50.0 mg/L. In yet another embodiment, the base media that has been exposed to UVC light further comprises phenylalanine, which is added to the UVC exposed base media to yield a concentration of about 25.0 to about 40.0 mg/L. In still another embodiment, the base media that has been exposed to UVC light further comprises phenylalanine, which is added to the UVC exposed base media to yield a concentration of about 30.0 to about 40.0 mg/L. In another embodiment, the base media that has been exposed to UVC light further comprises phenylalanine, which is added to the UVC exposed base media to yield a concentration of about 32.0 to about 38.0 mg/L. In yet another embodiment, the base media that has been exposed to UVC light further comprises phenylalanine, which is added to the UVC exposed base media to yield a concentration of about 34.0 to about 36.0 mg/L. In still another embodiment, the base media that has been exposed to UVC light further comprises phenylalanine, which is added to the UVC exposed base media to yield a concentration of about 35.0 mg/L. In another embodiment, the base media that has been exposed to UVC light further comprises phenylalanine, which is added to the UVC exposed base media to yield a concentration of about 36.0 mg/L. In yet another embodiment, the base media that has been exposed to UVC light further comprises phenylalanine, which is added to the UVC exposed base media to yield a concentration of about 35.5 mg/L.

In one embodiment, the base media that has been exposed to UVC light further comprises tryptophan, which is added to the UVC exposed base media to yield a concentration of about 0.1 to about 50.0 mg/L. In another embodiment, the base media that has been exposed to UVC light further comprises tryptophan, which is added to the UVC exposed base media to yield a concentration of about 1.0 to about 20.0 mg/L. In yet another embodiment, the base media that has been exposed to UVC light further comprises tryptophan, which is added to the UVC exposed base media to yield a concentration of about 5.0 to about 15.0 mg/L. In still another embodiment, the base media that has been exposed to UVC light further comprises tryptophan, which is added to the UVC exposed base media to yield a concentration of about 8.0 to about 12.0 mg/L. In another embodiment, the base media that has been exposed to UVC light further comprises tryptophan, which is added to the UVC exposed base media to yield a concentration of about 8.0 to about 10.0 mg/L. In another embodiment, the base media that has been exposed to UVC light further comprises tryptophan, which is added to the UVC exposed base media to yield a concentration of about 8.0 mg/L. In yet another embodiment, the base media that has been exposed to UVC light further comprises tryptophan, which is added to the UVC exposed base media to yield a concentration of about 8.5 mg/L. In still another embodiment, the base media that has been exposed to UVC light further comprises tryptophan, which is added to the UVC exposed base media to yield a concentration of about 9.0 mg/L. In another embodiment, the base media that has been exposed to UVC light further comprises tryptophan, which is added to the UVC exposed base media to yield a concentration of about 9.5 mg/L.

In one embodiment, the base media that has been exposed to UVC light further comprises tyrosine, which is added to the UVC exposed base media to yield a concentration of about 1.0 to about 100.0 mg/L. In another embodiment, the base media that has been exposed to UVC light further comprises tyrosine, which is added to the UVC exposed base media to yield a concentration of about 10.0 to about 90.0 mg/L. In another embodiment, the base media that has been exposed to UVC light further comprises tyrosine, which is added to the UVC exposed base media to yield a concentration of about 20.0 to about 80.0 mg/L. In yet another embodiment, the base media that has been exposed to UVC light further comprises tyrosine, which is added to the UVC exposed base media to yield a concentration of about 30.0 to about 70.0 mg/L. In still another embodiment, the base media that has been exposed to UVC light further comprises tyrosine, which is added to the UVC exposed base media to yield a concentration of about 40.0 to about 60.0 mg/L. In another embodiment, the base media that has been exposed to UVC light further comprises tyrosine, which is added to the UVC exposed base media to yield a concentration of about 50.0 to about 60.0 mg/L. In another embodiment, the base media that has been exposed to UVC light further comprises tyrosine, which is added to the UVC exposed base media to yield a concentration of about 53.0 to about 57.0 mg/L. In yet another embodiment, the base media that has been exposed to UVC light further comprises tyrosine, which is added to the UVC exposed base media to yield a concentration of about 54.0 to about 56.0 mg/L. In still another embodiment, the base media that has been exposed to UVC light further comprises tyrosine, which is added to the UVC exposed base media to yield a concentration of about 55.0 mg/L. In another embodiment, the base media that has been exposed to UVC light further comprises tyrosine, which is added to the UVC exposed base media to yield a concentration of about 55.5 mg/L. In yet another embodiment, the base media that has been exposed to UVC light further comprises tyrosine, which is added to the UVC exposed base media to yield a concentration of about 60.0 mg/L.

In one embodiment, the base media that has been exposed to UVC light further comprises lipoic acid, which is added to the UVC exposed base media to yield a concentration of about 0.01 to about 10.0 mg/L. In another embodiment, the base media that has been exposed to UVC light further comprises lipoic acid, which is added to the UVC exposed base media to yield a concentration of about 0.05 to about 5.0 mg/L. In yet another embodiment, the base media that has been exposed to UVC light further comprises lipoic acid, which is added to the UVC exposed base media to yield a concentration of about 0.08 to about 0.12 mg/L. In still another embodiment, the base media that has been exposed to UVC light further comprises lipoic acid, which is added to the UVC exposed base media to yield a concentration of about 0.100 to about 0.110 mg/L. In another embodiment, the base media that has been exposed to UVC light comprises lipoic acid, which is added to the UVC exposed base media to yield a concentration of about 0.101 to about 0.107 mg/L. In yet another embodiment, the base media that has been exposed to UVC light comprises lipoic acid, which is added to the UVC exposed base media to yield a concentration of about 0.102 to about 0.104 mg/L. In still another embodiment, the base media that has been exposed to UVC light comprises lipoic acid, which is added to the UVC exposed base media to yield a concentration of about 0.102 mg/L. In another embodiment, the base media that has been exposed to UVC light comprises lipoic acid, which is added to the UVC exposed base media to yield a concentration of about 0.103 mg/L. In yet another embodiment, the base media that has been exposed to UVC light comprises lipoic acid, which is added to the UVC exposed base media to yield a concentration of about 0.104 mg/L.

In one embodiment, the base media that has been exposed to UVC light comprises niacinamide, which is added to the UVC exposed base media to yield a concentration of about 0.1 to about 10.0 mg/L. In another embodiment, the base media that has been exposed to UVC light comprises niacinamide, which is added to the UVC exposed base media to yield a concentration of about 0.5 to about 5.0 mg/L. In yet another embodiment, the base media that has been exposed to UVC light comprises niacinamide, which is added to the UVC exposed base media to yield a concentration of about 1.0 to about 3.0 mg/L. In still another embodiment, the base media that has been exposed to UVC light comprises niacinamide, which is added to the UVC exposed base media to yield a concentration of about 1.5 to about 2.5 mg/L. In another embodiment, the base media that has been exposed to UVC light comprises niacinamide, which is added to the UVC exposed base media to yield a concentration of about 1.5 mg/L. In yet another embodiment, the base media that has been exposed to UVC light comprises niacinamide, which is added to the UVC exposed base media to yield a concentration of about 2.0 mg/L. In still another embodiment, the base media that has been exposed to UVC light comprises niacinamide, which is added to the UVC exposed base media to yield a concentration of about 2.5 mg/L.

In one embodiment, the base media that has been exposed to UVC light comprises pyridoxal, which is added to the UVC exposed base media to yield a concentration of about 0.1 to about 10.0 mg/L. In yet another embodiment, the base media that has been exposed to UVC light comprises pyridoxal, which is added to the UVC exposed base media to yield a concentration of about 0.5 to about 5.0 mg/L. In still another embodiment, the base media that has been exposed to UVC light comprises pyridoxal, which is added to the UVC exposed base media to yield a concentration of about 1.0 to about 3.0 mg/L. In another embodiment, the base media that has been exposed to UVC light comprises pyridoxal, which is added to the UVC exposed base media to yield a concentration of about 1.5 to about 2.5 mg/L. In yet another embodiment, the base media that has been exposed to UVC light comprises pyridoxal, which is added to the UVC exposed base media to yield a concentration of about 1.5 mg/L. In still another embodiment, the base media that has been exposed to UVC light comprises pyridoxal, which is added to the UVC exposed base media to yield a concentration of about 2.0 mg/L. In another embodiment, the base media that has been exposed to UVC light comprises pyridoxal, which is added to the UVC exposed base media to yield a concentration of about 2.5 mg/L.

In one embodiment, the base media that has been exposed to UVC light comprises pyridoxine, which is added to the UVC exposed base media to yield a concentration of about 0.0001 to about 1 mg/L. In another embodiment, the base media that has been exposed to UVC light comprises pyridoxine, which is added to the UVC exposed base media to yield a concentration of about 0.001 to about 0.1 mg/L. In yet another embodiment, the base media that has been exposed to UVC light comprises pyridoxine, which is added to the UVC exposed base media to yield a concentration of about 0.010 to about 0.050 mg/L. In still another embodiment, the base media that has been exposed to UVC light comprises pyridoxine, which is added to the UVC exposed base media to yield a concentration of about 0.020 to about 0.040 mg/L. In another embodiment, the base media that has been exposed to UVC light comprises pyridoxine, which is added to the UVC exposed base media to yield a concentration of about 0.025 to about 0.035 mg/L. In yet another embodiment, the base media that has been exposed to UVC light comprises pyridoxine, which is added to the UVC exposed base media to yield a concentration of about 0.030 to about 0.035 mg/L. In still another embodiment, the base media that has been exposed to UVC light comprises pyridoxine, which is added to the UVC exposed base media to yield a concentration of about 0.030 to about 0.032 mg/L. In another embodiment, the base media that has been exposed to UVC light comprises pyridoxine, which is added to the UVC exposed base media to yield a concentration of about 0.030 mg/L. In yet another embodiment, the base media that has been exposed to UVC light comprises pyridoxine, which is added to the UVC exposed base media to yield a concentration of about 0.031 mg/L. In still another embodiment, the base media that has been exposed to UVC light comprises pyridoxine, which is added to the UVC exposed base media to yield a concentration of about 0.032 mg/L.

In one embodiment, the base media that has been exposed to UVC light comprises riboflavin, which is added to the UVC exposed base media to yield a concentration of about 0.01 to about 10.0 mg/L. In another embodiment, the base media that has been exposed to UVC light comprises riboflavin, which is added to the UVC exposed base media to yield a concentration of about 0.05 to about 5.0 mg/L. In yet another embodiment, the base media that has been exposed to UVC light comprises riboflavin, which is added to the UVC exposed base media to yield a concentration of about 0.1 to about 1.0 mg/L. In still another embodiment, the base media that has been exposed to UVC light comprises riboflavin, which is added to the UVC exposed base media to yield a concentration of about 0.1 to about 0.5 mg/L. In another embodiment, the base media that has been exposed to UVC light comprises riboflavin, which is added to the UVC exposed base media to yield a concentration of about 0.1 to about 0.3 mg/L. In yet another embodiment, the base media that has been exposed to UVC light comprises riboflavin, which is added to the UVC exposed base media to yield a concentration of about 0.15 to about 0.25 mg/L. In still another embodiment, the base media that has been exposed to UVC light comprises riboflavin, which is added to the UVC exposed base media to yield a concentration of about 0.20 to about 0.23 mg/L. In another embodiment, the base media that has been exposed to UVC light comprises riboflavin, which is added to the UVC exposed base media to yield a concentration of about 0.21 to about 0.22 mg/L. In yet another embodiment, the base media that has been exposed to UVC light comprises riboflavin, which is added to the UVC exposed base media to yield a concentration of about 0.210 mg/L. In still another embodiment, the base media that has been exposed to UVC light comprises riboflavin, which is added to the UVC exposed base media to yield a concentration of about 0.213 mg/L. In another embodiment, the base media that has been exposed to UVC light comprises riboflavin, which is added to the UVC exposed base media to yield a concentration of about 0.216 mg/L. In yet another embodiment, the base media that has been exposed to UVC light comprises riboflavin, which is added to the UVC exposed base media to yield a concentration of about 0.219 mg/L.

In one embodiment, the base media that has been exposed to UVC light comprises thiamine, which is added to the UVC exposed base media to yield a concentration of about 0.1 to about 10.0 mg/L. In another embodiment, the base media that has been exposed to UVC light comprises thiamine, which is added to the UVC exposed base media to yield a concentration of about 0.5 to about 5.0 mg/L. In yet another embodiment, the base media that has been exposed to UVC light comprises thiamine, which is added to the UVC exposed base media to yield a concentration of about 1.0 to about 3.0 mg/L. In still another embodiment, the base media that has been exposed to UVC light comprises thiamine, which is added to the UVC exposed base media to yield a concentration of about 1.5 to about 2.5 mg/L. In another embodiment, the base media that has been exposed to UVC light comprises thiamine, which is added to the UVC exposed base media to yield a concentration of about 2.0 to about 2.3 mg/L. In yet another embodiment, the base media that has been exposed to UVC light comprises thiamine, which is added to the UVC exposed base media to yield a concentration of about 2.1 to about 2.2 mg/L. In still another embodiment, the base media that has been exposed to UVC light comprises thiamine, which is added to the UVC exposed base media to yield a concentration of about 2.0 mg/L. In another embodiment, the base media that has been exposed to UVC light comprises thiamine, which is added to the UVC exposed base media to yield a concentration of about 2.1 mg/L. In yet another embodiment, the base media that has been exposed to UVC light comprises thiamine, which is added to the UVC exposed base media to yield a concentration of about 2.2 mg/L.

In one embodiment, the base media that has been exposed to UVC light comprises methotrexate, which is added to the UVC exposed base media to yield a concentration of about 0.0000001 to about 0.001 g/L. In another embodiment, the base media that has been exposed to UVC light comprises methotrexate, which is added to the UVC exposed base media to yield a concentration of about 0.000001 to about 0.0001 g/L. In yet another embodiment, the base media that has been exposed to UVC light comprises methotrexate, which is added to the UVC exposed base media to yield a concentration of about 0.00001 to about 0.0001 g/L. In still another embodiment, the base media that has been exposed to UVC light comprises methotrexate, which is added to the UVC exposed base media to yield a concentration of about 0.00001 to about 0.00005 g/L. In another embodiment, the base media that has been exposed to UVC light comprises methotrexate, which is added to the UVC exposed base media to yield a concentration of about 0.00002 to about 0.00004 g/L.

In another embodiment, the base media that has been exposed to UVC light comprises methotrexate, which is added to the UVC exposed base media to yield a concentration of about 0.00003 g/L.

In one embodiment, the base media that has been exposed to UVC light comprises vitamin B12, which is added to the UVC exposed base media to yield a concentration of about 0.01 to about 10.0 mg/L. In another embodiment, the base media that has been exposed to UVC light comprises vitamin B12, which is added to the UVC exposed base media to yield a concentration of about 0.05 to about 5.0 mg/L. In yet another embodiment, the base media that has been exposed to UVC light comprises vitamin B12, which is added to the UVC exposed base media to yield a concentration of about 0.1 to about 1.0 mg/L. In still another embodiment, the base media that has been exposed to UVC light comprises vitamin B12, which is added to the UVC exposed base media to yield a concentration of about 0.3 to about 0.8 mg/L. In another embodiment, the base media that has been exposed to UVC light comprises vitamin B12, which is added to the UVC exposed base media to yield a concentration of about 0.5 to about 0.75 mg/L. In yet another embodiment, the base media that has been exposed to UVC light comprises vitamin B12, which is added to the UVC exposed base media to yield a concentration of about 0.6 to about 0.7 mg/L. In still another embodiment, the base media that has been exposed to UVC light comprises vitamin B12, which is added to the UVC exposed base media to yield a concentration of about 0.65 to about 0.70 mg/L. In another embodiment, the base media that has been exposed to UVC light comprises vitamin B12, which is added to the UVC exposed base media to yield a concentration of about 0.67 to about 0.69 mg/L. In yet another embodiment, the base media that has been exposed to UVC light comprises vitamin B12, which is added to the UVC exposed base media to yield a concentration of about 0.67 mg/L. In still another embodiment, the base media that has been exposed to UVC light comprises vitamin B12, which is added to the UVC exposed base media to yield a concentration of about 0.68 mg/L. In another embodiment, the base media that has been exposed to UVC light comprises vitamin B12, which is added to the UVC exposed base media to yield a concentration of about 0.69 mg/L.

### Cells, Proteins and Cell Culture

In general, cell culture media contains a base solution or “basal media,” referred to herein interchangeably as a “base media,” into which all of the desired components are added. In the context of the present invention, the “base media” refers to a cell culture media that does not comprise certain UV sensitive components. It will be understood that the base media described below may generally comprise UV sensitive components, but may optionally be formulated to not comprise certain UV sensitive components. For example, if RPMI 1640 is disclosed as a base media herein, it will be understood that it may optionally not comprise certain UV sensitive components that are otherwise typically found in RPMI 1640.

Typically, “cell culturing medium” (also called “culture medium,” “cell culture media,” or “tissue culture media”) is a term that is understood by the practitioner in the art and is known to refer to a nutrient solution in which cells, e.g., animal or mammalian cells, are grown and which generally provides at least one or more components from the following: an energy source (usually in the form of a carbohydrate such as glucose); all essential amino acids, and generally the twenty basic amino acids, plus cysteine; vitamins and/or other organic compounds typically required at low concentrations; lipids or free fatty acids, e.g., linoleic acid; and trace elements, e.g., inorganic compounds or naturally occurring elements that are typically required at very low concentrations, usually in the micromolar range. Cell culture medium can also be supplemented to contain a variety of optional components, such as hormones and other growth factors, e.g., insulin, transferrin, epidermal growth factor, serum, and the like; salts, e.g., calcium, magnesium and phosphate, and buffers, e.g., HEPES; nucleosides and bases, e.g., adenosine, thymidine, hypoxanthine; and protein and tissue hydrolysates, e.g., hydrolyzed animal protein (peptone or peptone mixtures, which can be obtained from animal byproducts, purified gelatin or plant material); antibiotics, e.g., gentamycin; and cell protective agents, e.g., a Pluronic polyol (Pluronic F68). Cell culture medium, in certain embodiments, is serum-free and free of products or ingredients of animal origin.

As is appreciated by the practitioner, animal or mammalian cells are cultured in a medium suitable for the particular cells being cultured and which can be determined by the person of skill in the art without undue experimentation. Commercially available media can be utilized and include, but is not limited to, Iscove's Modified Dulbecco's Medium, RPMI 1640, Minimal Essential Medium-alpha. (MEM-alpha), Dulbecco's Modification of Eagle's Medium (DMEM), DME/F12, alpha MEM, Basal Medium Eagle with Earle's BSS, DMEM high Glucose, with Glutamine, DMEM high glucose, without Glutamine, DMEM low Glucose, without Glutamine, DMEM:F12 1:1, with Glutamine, GMEM (Glasgow's MEM), GMEM with glutamine, Grace's Complete Insect Medium, Grace's Insect Medium, without FBS, Ham's F-10, with Glutamine, Ham's F-12, with Glutamine, IMDM with HEPES and Glutamine, IMDM with HEPES and without Glutamine, IP41 Insect Medium, 15 (Leibovitz) (2×), without Glutamine or Phenol Red, 15 (Leibovitz), without Glutamine, McCoy's 5A Modified Medium, Medium 199, MEM Eagle, without Glutamine or Phenol Red (2×), MEM Eagle-Earle's BSS, with glutamine, MEM Eagle-Earle's BSS, without Glutamine, MEM Eagle-Hanks BSS, without Glutamine, NCTC-109, with Glutamine, Richter's CM Medium, with Glutamine, RPMI 1640 with HEPES, Glutamine and/or Penicillin-Streptomycin, RPMI 1640, with Glutamine, RPMI 1640, without Glutamine, Schneider's Insect Medium or any other media known to one skilled in the art, which are formulated for particular cell types. To the foregoing exemplary media can be added supplementary components or ingredients, including optional components, in appropriate concentrations or amounts, as necessary or desired, and as would be known and practiced by those having in the art using routine skill.

In addition, cell culture conditions suitable for the methods of the present invention are those that are typically employed and known for batch, fed-batch, or continuous culturing of cells, with attention paid to pH, dissolved oxygen (O₂), and carbon dioxide (CO₂), agitation and humidity, and temperature.

The compositions of the present invention can be used to culture a variety of cells. In one embodiment, the medium is used to culture eukaryotic cells such as plant and/or animal cells. The cells can be mammalian cells, fish cells, insect cells, amphibian cells or avian cells. The medium can be used to culture cells including, but not limited to, MK2.7 cells, PER-C6 cells, CHO cells, HEK 293 cells, COS cells and Sp2/0 cells, 5L8 hybridoma cells, Daudi cells, EL4 cells, HeLa cells, HL-60 cells, K562 cells, Jurkat cells, THP-1 cells, Sp2/0 cells, primary epithelial cells (e.g., keratinocytes, cervical epithelial cells, bronchial epithelial cells, tracheal epithelial cells, kidney epithelial cells and retinal epithelial cells) and established cell lines and their strains (e.g., 293 embryonic kidney cells, BHK cells, HeLa cervical epithelial cells and PER-C6 retinal cells, MDBK (NBL-1) cells, 911 cells, CRFK cells, MDCK cells, BeWo cells, Chang cells, Detroit 562 cells, HeLa 229 cells, HeLa S3 cells, Hep-2 cells, KB cells, LS 180 cells, LS 174T cells, NCI-H-548 cells, RPMI 2650 cells, SW-13 cells, T24 cells, WI-28 VA13, 2RA cells, WISH cells, BS-C-I cells, LLC-MK₂ cells, Clone M-3 cells, 1-10 cells, RAG cells, TCMK-1 cells, Y-1 cells, LLC-PK₁ cells, PK(15) cells, GH₁ cells, GH₃ cells, L2 cells, LLC-RC 256 cells, MH₁C₁ cells, XC cells, MDOK cells, VSW cells, and TH-1, B1 cells, or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, MiCl₁ cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C₃H/IOTI/2 cells, HSDM₁C₃ cells, KLN₂O₅ cells, McCoy cells, Mouse L cells, Strain 2071 (Mouse L) cells, L-M strain (Mouse L) cells, L-MTK (Mouse L) cells, NCTC clones 2472 and 2555, SCC-PSA1 cells, Swiss/3T3 cells, Indian muntac cells, SIRC cells, C_{II} cells, and Jensen cells, or derivatives thereof) or any other cell type known to one skilled in the art.

The media disclosed herein can be used to culture cells in suspension or adherent cells. The compositions of the present invention are suitable for either adherent, monolayer or suspension culture, transfection, and cultivation of cells, and for expression of proteins or antibodies in cells in monolayer or suspension culture.

Cells supported by the medium of the present invention can be derived from any animal, such as a mouse or a hamster or a human. The cells cultivated in the present media can be normal cells or abnormal cells (i.e., transformed cells, established cells, or cells derived from diseased tissue samples).

Animal cells, mammalian cells, cultured cells, animal or mammalian host cells, host cells, recombinant cells, recombinant host cells, and the like, are all terms for the cells that can be cultured according to the processes of this invention. Such cells are typically cell lines obtained or derived from mammals and are able to grow and survive when placed in either monolayer culture or suspension culture in medium containing appropriate nutrients and/or growth factors. Growth factors and nutrients that are necessary for the growth and maintenance of particular cell cultures are able to be readily determined empirically by those having skill in the pertinent art, such as is described, for example, by Barnes and Sato, (1980, Cell, 22:649); in Mammalian Cell Culture, Ed. J. P. Mather, Plenum Press, NY, 1984; and in U.S. Pat. No. 5,721,121.

Numerous types of cells can be cultured according to the methods of the present invention. The cells are typically animal or mammalian cells that can express and secrete, or that can be molecularly engineered to express and secrete, large quantities of a particular protein, more particularly, a glycoprotein of interest, into the culture medium. It will be understood that the glycoprotein produced by a host cell can be endogenous or homologous to the host cell. Alternatively, the glycoprotein is heterologous, i.e., foreign, to the host cell, for example, a human glycoprotein produced and secreted by a Chinese hamster ovary (CHO) host cell. Additionally, mammalian glycoproteins, i.e., those originally obtained or derived from a mammalian organism, are attained by the methods the present invention and can be secreted by the cells into the culture medium.

Nonlimiting examples of mammalian glycoproteins that can be advantageously produced by the methods of this invention include cytokines, cytokine receptors, growth factors (e.g., EGF, HER-2, FGF-α, FGF-β, TGF-α, TGF-β, PDGF. IGF-1, IGF-2, NGF, NGF-β); growth factor receptors, including fusion or chimeric proteins. Other nonlimiting examples include growth hormones (e.g., human growth hormone, bovine growth hormone); insulin (e.g., insulin A chain and insulin B chain), proinsulin; erythropoietin (EPO); darbepoetin, colony stimulating factors (e.g., G-CSF, GM-CSF, M-CSF); interleukins (e.g., IL-1 through IL-12); vascular endothelial growth factor (VEGF) and its receptor (VEGF-R); interferons (e.g., IFN-α, β, or γ); tumor necrosis factor (e.g., TNF-α and TNF-β) and their receptors, TNFR-1 and TNFR-2; thrombopoietin (TPO); thrombin; brain natriuretic peptide (BNP); clotting factors (e.g., Factor VIII, Factor IX, von Willebrands factor, and the like); anti-clotting factors; tissue plasminogen activator (TPA), e.g., urokinase or human urine or tissue type TPA; follicle stimulating hormone (FSH); luteinizing hormone (LH); calcitonin; CD proteins (e.g., CD3, CD4, CD8, CD28, CD19, etc.); CTLA proteins (e.g., CTLA4); T-cell and B-cell receptor proteins; bone morphogenic proteins (BNPs, e.g., BMP-1, BMP-2, BMP-3, etc.); neurotrophic factors, e.g., bone derived neurotrophic factor (BDNF); neurotrophins, e.g., 3-6; renin; rheumatoid factor; RANTES; albumin; relaxin; macrophage inhibitory protein (e.g., MIP-1, MIP-2); viral proteins or antigens; surface membrane proteins; ion channel proteins; enzymes; regulatory proteins; antibodies; immunomodulatory proteins, (e.g., HLA, MHC, the B7 family); homing receptors; transport proteins; superoxide dismutase (SOD); G-protein coupled receptor proteins (GPCRs); neuromodulatory proteins; Alzheimer's Disease associated proteins and peptides, (e.g., A-beta), and others as known in the art. Fusion proteins and polypeptides, chimeric proteins and polypeptides, as well as fragments or portions, or mutants, variants, or analogs of any of the aforementioned proteins and polypeptides are also included among the suitable proteins, polypeptides and peptides that can be produced by the methods of the present invention.

Nonlimiting examples of animal or mammalian host cells suitable for harboring, expressing, and producing proteins for subsequent isolation and/or purification include Chinese hamster ovary cells (CHO), such as CHO-K1 (ATCC CCL-61), DG44 (Chasin et al., 1986, Som. Cell Molec. Genet., 12:555-556; Kolkekar et al., 1997, Biochemistry, 36:10901-10909; and WO 01/92337 A2), dihydrofolate reductase negative CHO cells (CHO/-DHFR, Urlaub and Chasin, 1980, Proc. Natl. Acad. Sci. USA, 77:4216), and dp12.CHO cells (U.S. Pat. No. 5,721,121); monkey kidney CV1 cells transformed by SV40 (COS cells, COS-7, ATCC CRL-1651); human embryonic kidney cells (e.g., 293 cells, or 293 cells subcloned for growth in suspension culture, Graham et al., 1977, J. Gen. Virol., 36:59); baby hamster kidney cells (BHK, ATCC CCL-10); monkey kidney cells (CV1, ATCC CCL-70); African green monkey kidney cells (VERO-76, ATCC CRL-1587; VERO, ATCC CCL-81); mouse sertoli cells (TM4, Mather, 1980, Biol. Reprod., 23:243-251); human cervical carcinoma cells (HELA, ATCC CCL-2); canine kidney cells (MDCK, ATCC CCL-34); human lung cells (W138, ATCC CCL-75); human hepatoma cells (HEP-G2, HB 8065); mouse mammary tumor cells (MMT 060562, ATCC CCL-51); buffalo rat liver cells (BRL 3A, ATCC CRL-1442); TR1 cells (Mather, 1982, Annals NY Acad. Sci., 383:44-68); MCR 5 cells; FS4 cells.

The cells suitable for culturing using the methods and processes of the present invention can contain introduced, e.g., via transformation, transfection, infection, or injection, expression vectors (constructs), such as plasmids and the like, that harbor coding sequences, or portions thereof, encoding the proteins for expression and production in the culturing process. Such expression vectors contain the necessary elements for the transcription and translation of the inserted coding sequence. Methods which are well known to and practiced by those skilled in the art can be used to construct expression vectors containing sequences encoding the produced proteins and polypeptides, as well as the appropriate transcriptional and translational control elements. These methods include in vitro recombinant DNA techniques, synthetic techniques, and in vivo genetic recombination. Such techniques are described in J. Sambrook et al., 1989, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y., all of which are incorporated herein for any purpose.

In addition to cultivation of mammalian cells in suspension or in monolayer cultures, the present media and methods may be used in methods for producing viruses from mammalian cells. Such methods according to this aspect of the invention comprise (a) obtaining a mammalian cell to be infected with a virus; (b) contacting the cell with a virus under conditions suitable to promote the infection of the cell by the virus; and (c) cultivating the cell in the culture media of the invention under conditions suitable to promote the production of virus by the cell. According to the invention, the cell may be contacted with the virus either prior to, during or following cultivation of the cell in the culture media of the invention; optimal methods for infecting a mammalian cell with a virus are well-known in the art and will be familiar to one of ordinary skill. Virus-infected mammalian cells cultivated in suspension in the media of the invention may be expected to produce higher virus titers (e.g., 2-, 3-, 5-, 10-, 20-, 25-, 50-, 100-, 250-, 500-, or 1000-fold higher titers) than those cells not cultivated in suspension in the media of the invention. These methods may be used to produce a variety of mammalian viruses and viral vectors, including but not limited to adenoviruses, adeno-associated viruses, retroviruses, influenza viruses and other viruses for vaccine manufacture and the like. Following cultivation of the infected cells in the present media, the used culture media comprising viruses, viral vectors, viral particles or components thereof (proteins and/or nucleic acids (DNA and/or RNA)) may be used for a variety of purposes, including vaccine production, production of viral vectors for use in cell transfection or gene therapy, infection of animals or cell cultures, study of viral proteins and/or nucleic acids and the like. Alternatively, viruses, viral vectors, viral particles or components thereof may optionally be isolated from the used culture medium according to techniques for protein and/or nucleic acid isolation that will be familiar to one of ordinary skill in the art.

### Types of Cell Cultures

For the purposes of understanding, yet without limitation, it will be appreciated by the skilled practitioner that cell cultures and culturing runs for protein production can include three general types; namely, continuous culture, batch culture and fed-batch culture. In a continuous culture, for example, fresh culture medium supplement (i.e., feeding medium) is provided to the cells during the culturing period, while old culture medium is removed daily and the product is harvested, for example, daily or continuously. In continuous culture, feeding medium can be added daily and can be added continuously, i.e., as a drip or infusion. For continuous culturing, the cells can remain in culture as long as is desired, so long as the cells remain alive and the environmental and culturing conditions are maintained.

In batch culture, cells are initially cultured in medium and this medium is not removed, replaced, or supplemented, i.e., the cells are not “fed” with new medium, during or before the end of the culturing run. The desired product is harvested at the end of the culturing run.

For fed-batch cultures, the culturing run time is increased by supplementing the culture medium one or more times daily (or continuously) with fresh medium during the run, i.e., the cells are “fed” with new medium (“feeding medium”) during the culturing period. Fed-batch cultures can include the various feeding regimens and times as described above, for example, daily, every other day, every two days, etc., more than once per day, or less than once per day, and so on. Further, fed-batch cultures can be fed continuously with feeding medium. The desired product is then harvested at the end of the culturing/production run.

According to the present invention, cell culture can be carried out, and glycoproteins can be produced by cells, under conditions for the large or small scale production of proteins, using culture vessels and/or culture apparatuses that are conventionally employed for animal or mammalian cell culture. As is appreciated by those having skill in the art, tissue culture dishes, T-flasks and spinner flasks are typically used on a laboratory scale. For culturing on a larger scale (e.g., 500 L, 1000 L, 2000 L, 5000 L, 10000 L and the like), procedures including, but not limited to, a fermentor type tank culture device, an air lift type culture device, a fluidized bed bioreactor, a hollow fiber bioreactor, roller bottle culture, a stirred tank bioreactor systems, a packed bed type culture device, or any other suitable devise known to one skilled in the art can be used. Microcarriers may or may not be used with the roller bottle or stirred tank bioreactor systems. The systems can be operated in a batch, continuous, or fed-batch mode. In addition, the culture apparatus or system may or may not be equipped with a cell separator using filters, gravity, centrifugal force, and the like.

In one embodiment, the cell culture base media is exposed to UVC light and then stored until needed, at which time, the additive package will be added to the UVC exposed base media prior to use in cell culture. In another embodiment, the cell culture base media is treated as part of an automated process, wherein the additive package is added to the UVC exposed base media as part of a stepwise process with no breaks in time for storage. The skilled practitioner will readily envision any number of ways this UVC exposure process can occur, with the necessary step being UVC exposure of a base media, followed at some point by the addition of an additive package.

### UV Light

The term “ultraviolet light” refers to a section of the electromagnetic spectrum of light extending from the x-ray region (100 nm) to the visible region (400 nm). In particular, ultraviolet light is generally divided into four fractions: (1) vacuum ultraviolet light—having a wavelength of 100 to 200 nm, (2) ultraviolet C (UVC)—having a wavelength of 200 to 280 nm, (3) ultraviolet B (UVB)—having a wavelength of 280 to 315 nm, and (4) ultraviolet A (UVA)—having a wavelength of 315 to 400 nm. In one embodiment, cell culture base media is exposed to UVC light prior to introducing the cell culture media into the culture apparatus (e.g., a bioreactor).

In one embodiment of the invention, cell culture media is exposed to UVC light having a wavelength of between 200 nm and 280 nm prior to introducing the cell culture media into a bioreactor. In another embodiment, cell culture media is exposed to UVC light having a wavelength of between 200 nm and 275 nm. In a further embodiment, cell culture media is exposed to UVC light having a wavelength of between 200 nm and 270 nm. In yet another embodiment, cell culture media is exposed to UVC light having a wavelength of between 200 nm and 265 nm. In still a further embodiment, cell culture media is exposed to UVC light having a wavelength of between 200 nm and 260 nm. In another embodiment, cell culture media is exposed to UVC light having a wavelength of between 200 nm and 255 nm. In a further embodiment, cell culture media is exposed to UVC light having a wavelength of between 200 nm and 250 nm. In yet another embodiment, cell culture media is exposed to UVC light having a wavelength of between 200 nm and 245 nm. In still another embodiment, cell culture media is exposed to UVC light having a wavelength of between 200 nm and 240 nm. In a further embodiment, cell culture media is exposed to UVC light having a wavelength of between 200 nm and 235 nm. In one embodiment, cell culture media is exposed to UVC light having a wavelength of between 200 nm and 230 nm. In another embodiment, cell culture media is exposed to UVC light having a wavelength of between 200 nm and 225 nm. In yet another embodiment, cell culture media is exposed to UVC light having a wavelength of between 200 nm and 220 nm. In still another embodiment, cell culture media is exposed to UVC light having a wavelength of between 200 nm and 215 nm. In yet another embodiment, cell culture media is exposed to UVC light having a wavelength of between 200 nm and 210 nm. In a further embodiment, cell culture media is exposed to UVC light having a wavelength of between 200 nm and 205 nm. In yet a further embodiment, cell culture media is exposed to UVC light having a wavelength of between 205 nm and 280 nm. In a still a further embodiment, cell culture media is exposed to UVC light having a wavelength of between 210 nm and 280 nm. In yet a further embodiment, cell culture media is exposed to UVC light having a wavelength of between 215 nm and 280 nm. In still a further embodiment, cell culture media is exposed to UVC light having a wavelength of between 220 nm and 280 nm. In another embodiment, cell culture media is exposed to UVC light having a wavelength of between 225 nm and 280 nm. In a further embodiment, cell culture media is exposed to UVC light having a wavelength of between 230 nm and 280 nm. In still another embodiment, cell culture media is exposed to UVC light having a wavelength of between 235 nm and 280 nm. In another embodiment, cell culture media is exposed to UVC light having a wavelength of between 240 nm and 280 nm. In yet a further embodiment, cell culture media is exposed to UVC light having a wavelength of between 245 nm and 280 nm. In another embodiment, cell culture media is exposed to UVC light having a wavelength of between 250 nm and 280 nm. In a further embodiment, cell culture media is exposed to UVC light having a wavelength of between 255 nm and 280 nm. In still a further embodiment, cell culture media is exposed to UVC light having a wavelength of between 260 nm and 280 nm. In one embodiment, cell culture media is exposed to UVC light having a wavelength of between 265 nm and 280 nm. In yet another embodiment, cell culture media is exposed to UVC light having a wavelength of between 270 nm and 280 nm. In still another embodiment, cell culture media is exposed to UVC light having a wavelength of between 275 nm and 280 nm. In yet another embodiment, cell culture media is exposed to UVC light having a wavelength of between 205 nm and 275 nm. In another embodiment, cell culture media is exposed to UVC light having a wavelength of between 210 nm and 270 nm. In still a further embodiment, cell culture media is exposed to UVC light having a wavelength of between 215 nm and 265 nm. In another embodiment, cell culture media is exposed to UVC light having a wavelength of between 220 nm and 260 nm. In still another embodiment, cell culture media is exposed to UVC light having a wavelength of between 225 nm and 255 nm. In yet another embodiment, cell culture media is exposed to UVC light having a wavelength of between 230 nm and 250 nm. In a further embodiment, cell culture media is exposed to UVC light having a wavelength of between 235 nm and 245 nm. In yet another embodiment, cell culture media is exposed to UVC light having a wavelength of between 245 nm and 265 nm. In another embodiment, cell culture media is exposed to UVC light having a wavelength of between 248 nm and 260 nm. In yet a further embodiment, cell culture media is exposed to UVC light having a wavelength of between 249 nm and 259 nm. In a further embodiment, cell culture media is exposed to UVC light having a wavelength of between 250 nm and 258 nm. In still a further embodiment, cell culture media is exposed to UVC light having a wavelength of between 251 nm and 257 nm. In yet another embodiment, cell culture media is exposed to UVC light having a wavelength of between 252 nm and 256 nm. In another embodiment, cell culture media is exposed to UVC light having a wavelength of between 253 nm and 255 nm.

In another embodiment of the invention, cell culture media is exposed to UVC light having a wavelength of 254 nm prior to introducing the cell culture media into a bioreactor. In other embodiments of the invention, cell culture media is exposed to UVC light having a wavelength of 254 nm+/−1 nm, or a wavelength of 254 nm+/−2 nm, or a wavelength of 254 nm+/−3 nm, or a wavelength of 254 nm+/−4 nm, or a wavelength of 254 nm+/−5 nm, or a wavelength of 254 nm+/−6 nm, or a wavelength of 254 nm+/−7 nm, or a wavelength of 254 nm+/−8 nm, or a wavelength of 254 nm+/−9 nm, or a wavelength of 254 nm+/−10 nm, or a wavelength of 254 nm+/−15 nm, or a wavelength of 254 nm+/−20 nm, or a wavelength of 254 nm+/−25 nm.

The term “energy,” as used herein, refers to the amount of ultraviolet radiation in milliJoules/cm² or Joules/meters² to which treated cell culture media is exposed. In one embodiment of the invention, cell culture media is exposed to UVC light at an energy density of 25-350 mJ/cm², more preferably at an energy density of 60-250 mJ/cm², and most preferably at an energy density of 100-150 mJ/cm², prior to introducing the cell culture media into a bioreactor. In another embodiment, cell culture media is exposed to UVC light at an energy density of 125 mJ/cm² prior to introducing the cell culture media into a bioreactor. In other embodiments of the invention, the cell culture media is exposed to UVC light at an energy density of 125 mJ/cm²+/−1 mJ/cm², or at an energy density of 125 mJ/cm²+/−2 mJ/cm², or at an energy density of 125 mJ/cm²+/−3 mJ/cm², or at an energy density of 125 mJ/cm²+/−4 mJ/cm², or at an energy density of 125 mJ/cm²+/−5 mJ/cm², or at an energy density of 125 mJ/cm²+/−10 mJ/cm², or at an energy density of 125 mJ/cm²+/−15 mJ/cm², or at an energy density of 125 mJ/cm²+/−20 mJ/cm², or at an energy density of 125 mJ/cm²+/−25 mJ/cm², or at an energy density of 125 mJ/cm²+/−30 mJ/cm², or at an energy density of 125 mJ/cm²+/−40 mJ/cm², or at an energy density of 125 mJ/cm²+/−50 mJ/cm², or at an energy density of 125 mJ/cm²+/−60 mJ/cm², or at an energy density of 125 mJ/cm²+/−70 mJ/cm², or at an energy density of 125 mJ/cm²+/−80 mJ/cm², or at an energy density of 125 mJ/cm²+/−90 mJ/cm², or at an energy density of 125 mJ/cm²+/−100 mJ/cm², or at an energy density of 125 mJ/cm²+/−110 mJ/cm², or at an energy density of 125 mJ/cm²+/−120 mJ/cm². In yet other embodiments of the invention, the cell culture mediate is exposed to UVC light at an energy density of 175 mJ/cm²+/−1 mJ/cm², or at an energy density of 175 mJ/cm²+/−2 mJ/cm², or at an energy density of 175 mJ/cm²+/−3 mJ/cm², or at an energy density of 175 mJ/cm²+/−4 mJ/cm², or at an energy density of 175 mJ/cm²+/−5 mJ/cm², or at an energy density of 175 mJ/cm²+/−10 mJ/cm², or at an energy density of 175 mJ/cm²+/−15 mJ/cm², or at an energy density of 175 mJ/cm²+/−20 mJ/cm², or at an energy density of 175 mJ/cm²+/−25 mJ/cm², or at an energy density of 175 mJ/cm²+/−30 mJ/cm², or at an energy density of 175 mJ/cm²+/−40 mJ/cm², or at an energy density of 175 mJ/cm²+/−50 mJ/cm², or at an energy density of 175 mJ/cm²+/−60 mJ/cm², or at an energy density of 175 mJ/cm²+/−70 mJ/cm² or at an energy density of 175 mJ/cm²+/−80 mJ/cm², or at an energy density of 175 mJ/cm²+/−90 mJ/cm², or at an energy density of 175 mJ/cm²+/−100 mJ/cm², or at an energy density of 175 mJ/cm²+/−110 mJ/cm², or at an energy density of 175 mJ/cm²+/−120 mJ/cm², or at an energy density of 175 mJ/cm²+/−130 mJ/cm², or at an energy density of 175 mJ/cm²+/−140 mJ/cm², or at an energy density of 175 mJ/cm²+/−150 mJ/cm², or at an energy density of 175 mJ/cm²+/−160 mJ/cm², or at an energy density of 175 mJ/cm²+/−170 mJ/cm².

While specific examples of energy densities have been provided, it is noted that a UVC source that can be used in the methods disclosed herein delivers a range of energy densities around a target energy density; this range of energy densities is also referred to as a “dose distribution.” One feature of an energy density distribution is the asymmetry of the spread of energy densities, which possesses a “high dose tail.” The distribution can be abstracted using the probability measures of P10, P50, P90, and mean. The P# values describe the dose value at which #% of the fluid (e.g., media) is treated. Thus, a P10 value of 60 mJ/cm² means that 10% of the fluid received an energy density of less than 60 mJ/cm². This approach is suitable for UVC exposure devices, such as the helical UVC reactors described herein.

In another embodiment of the disclosed methods, a laminar UVC exposure device (e.g., a thin film UVC reactor) can be employed to deliver UVC light. When a laminar or thin-film UVC exposure device is employed the distribution can be approximated by P10=1/2*Mean, P90=2*Mean. Accordingly, if a laminar or thin film UVC exposure device were to be employed under the conditions described herein the distribution would be P10˜0, Mean=125 mJ/cm² and P90˜250 mJ/cm².

Consistent with the above discussion, it is noted that when a target energy density of UVC light is provided in the instant disclosure, it is implicit that the target energy density embodies a range of dosages that are described by the dose distribution rules provided above. More specifically, in one embodiment a target UVC energy density includes all energy densities within the P10-P60 distribution of the energy density; in another embodiment a target UVC energy densities includes all energy densities within the P65 distribution of the energy density; in another embodiment a target UVC energy density includes all energy densities within the P70 distribution of the energy density; in another embodiment a target UVC energy density includes all energy densities within the P75 distribution of the dose; in another embodiment a target UVC energy density includes all energy densities within the P80 distribution of the energy density; in another embodiment a target UVC dose includes all energy densities within the P85 distribution of the energy density; in another embodiment a target UVC energy density includes all energy densities within the P90 distribution of the energy density; in another embodiment a target UVC energy density includes all energy densities within the P95 distribution of the energy density.

In the context of the instant disclosure it will be understood that a UVC energy density can be described using the scalar ranges provided (e.g., an energy density of 125 mJ/cm²+/−1 mJ/cm², or an energy density of 125 mJ/cm²+/−2 mJ/cm², or an energy density of 125 mJ/cm²+/−3 mJ/cm², or an energy density of 125 mJ/cm²+/−4 mJ/cm², or an energy density of 125 mJ/cm²+/−5 mJ/cm², or an energy density of 125 mJ/cm²+/−10 mJ/cm², or an energy density of 125 mJ/cm²+/−15 mJ/cm², or an energy density of 125 mJ/cm²+/−20 mJ/cm², or an energy density of 125 mJ/cm²+/−25 mJ/cm², or an energy density of 125 mJ/cm²+/−30 mJ/cm², or an energy density of 125 mJ/cm²+/−40 mJ/cm², or an energy density of 125 mJ/cm²+/−50 mJ/cm², or an energy density of 125 mJ/cm²+/−60 mJ/cm², or an energy density of 125 mJ/cm²+/−70 mJ/cm², or an energy density of 125 mJ/cm²+/−80 mJ/cm², or an energy density of 125 mJ/cm²+/−90 mJ/cm², or an energy density of 125 mJ/cm²+/−100 mJ/cm², or an energy density of 125 mJ/cm²+/−110 mJ/cm², or an energy density of 125 mJ/cm²+/−120 mJ/cm² or in yet other of the invention, an energy density of 175 mJ/cm²+/−1 mJ/cm², or an energy density of 175 mJ/cm²+/−2 mJ/cm², or an energy density of 175 mJ/cm²+/−3 mJ/cm², or an energy density of 175 mJ/cm²+/−4 mJ/cm², or an energy density of 175 mJ/cm²+/−5 mJ/cm², or an energy density of 175 mJ/cm²+/−10 mJ/cm², or an energy density of 175 mJ/cm²+/−15 mJ/cm², or an energy density of 175 mJ/cm²+/−20 mJ/cm², or an energy density of 175 mJ/cm²+/−25 mJ/cm², or an energy density of 175 mJ/cm²+/−30 mJ/cm², or an energy density of 175 mJ/cm²+/−40 mJ/cm², or an energy density of 175 mJ/cm²+/−50 mJ/cm², or an energy density of 175 mJ/cm²+/−60 mJ/cm², or an energy density of 175 mJ/cm²+/−70 mJ/cm² or an energy density of 175 mJ/cm²+/−80 mJ/cm², or an energy density of 175 mJ/cm²+/−90 mJ/cm², or an energy density of 175 mJ/cm²+/−100 mJ/cm², or an energy density of 175 mJ/cm²+/−110 mJ/cm², or an energy density of 175 mJ/cm²+/−120 mJ/cm², or an energy density of 175 mJ/cm²+/−130 mJ/cm², or an energy density of 175 ml/cm²+/−140 mJ/cm², or an energy density of 175 mJ/cm²+/−150 mJ/cm², or an energy density of 175 mJ/cm²+/−160 mJ/cm², or an energy density of 175 mJ/cm²+/−170 mJ/cm²), or it can be described using the dosage distribution function described (e.g., energy densities within the ranges of P10-60, P65, P70, P75, P80, P85, P90 and P95 of the target energy density).

### UVC Exposure Devices

The cell culture media and methods of the invention can be used with any device, system, apparatus or method to expose the cell culture base media to UVC. Nonlimiting examples of devices can be found in U.S. Pat. No. 7,651,660, U.S. Pat. No. 6,773,608, U.S. Pat. No. 6,596,230, U.S. Pat. No. 5,725,757, U.S. Pat. No. 5,707,594 and U.S. Pat. Appl. Publ. No. 20040245164A1. In one embodiment, the cell culture media and methods of the invention are used with a helical UV reactor. In another embodiment, the cell culture media and methods of the invention are used with a laminar flow (or thin film) UV reactor.

### Other Media Treatments

It is contemplated that the cell culture media and methods provided herein can be used with treatment methods or devices other than UVC exposure to sterilize or disinfect media prior to cell culture. In one embodiment, HTST is used in combination with the media and methods of the present invention (see, e.g., U.S. Pat. No. 7,420,183). In another embodiment, adjustment of pH (e.g., to below 4) is used in combination with the media and methods of the present invention.

In one embodiment, cell culture media is subjected to filtration step after being exposed to UVC light. As used herein, the terms “sterile filtration” and “sterile filter” refer to the removal of micro plasma and other potential contaminants from cell culture media through use of a standard biological sterile filter. In one embodiment of the invention, cell culture media is passed through a sterile filter having pores with a maximum size of 200 nm prior to introducing the cell culture media into a bioreactor.

In another embodiment, cell culture media is passed through a depth filter prior to or following exposure to UVC light. The term “depth filter” refers to a filter that has multiple filtration layers, each layer being responsible for the filtration of particulate matter of different sizes and densities. This type of filtration process is similar to size exclusion. Light material is isolated at the top of the filter bed. The media becomes progressively finer and denser in the lower layers. Larger suspended particles are removed in the upper layers, while smaller particles are removed by lower layers.

In another embodiment, the cell culture media is treated with chemicals that inactivate viruses, such as solvents, detergents, psoralen, or beta-propiolactone.

### Additional Embodiments

In one embodiment, the instant disclosure provides a method providing virus-inactivated media by supplementing the media with excess quantities of media components known or suspected to be susceptible to UVC light, e.g., lipoic acid, histidine, phenylalanine, tryptophan, tyrosine, folic acid, niacinamide, pyridoxal, pyridoxine, riboflavin, thiamine, methotrexate, and vitamin B12 (cyanacobalamin), to compensate for their partial destruction upon treatment with UVC light.

As described herein, various common media components degrade or become biologically inactive following exposure to UVC light. In order to compensate for this degradation, media components susceptible to UVC-mediated degradation can be added to a media recipe in surplus amounts which provide adequate amounts of these compounds following exposure to UVC light. In other words, the amount of a given media component that is inactivated by UVC exposure can be supplemented by the addition of the amount of the inactivated component so that the media has the requisite amount of the component. In one embodiment the component can be added as a supplement to the media prior to UVC exposure, while in another embodiment the component can be added as a supplement to the media following UVC exposure.

In a particular example, a media component known or suspected to be inactivated by UVC light is identified. The initial amount of the component in the media is then identified. Separately, an empirical determination is made of the amount of the component that is degraded based on an intended UVC dose. Alternatively, the determination can be made by calculation, using known properties of the component as a guide (e.g., absorbance, concentration, UVC dose, etc). The amount of the component that is predicted to be degraded is then added to the media in a sterile addition (e.g., a sterile solution comprising the component in the appropriate concentration). Subsequently, the supplemented media is exposed to UVC light.

In another particular example, a media component known or suspected to be inactivated by UVC light is identified. The initial amount of the component in the media is then identified. Separately, an empirical determination is made of the amount of the component that is degraded based on an intended UVC dose. Alternatively, the determination can be made by calculation, using known properties of the component as a guide (e.g., absorbance, concentration, UVC dose, etc). The supplemented media is then exposed to UVC light. Subsequently, the amount of the component that is predicted to be degraded is then added to the media in a sterile addition (e.g., a sterile solution comprising the component in the appropriate concentration).

The invention having been described, the following examples are offered by way of illustration, and not limitation.

### EXAMPLES

### Materials and Methods for all Examples

Recombinant human erythropoietin was produced from CHO cell lines cultured in DMEM/F12 base media comprising the UV sensitive components lipoic acid, histidine, phenylalanine, tryptophan, tyrosine, folic acid, niacinamide, pyridoxal, pyridoxine, riboflavin, thiamine, and vitamin B12, and methotrexate.

The UVC treated and control medium for these experiments was run through a sterilizing grade filter. Cells were thawed from a vial and put through scale-up in expanded volume roller bottles, followed by inoculation into a series of bioreactors for the production phase of the process.

These experiments were carried through three harvests at the following intervals: Harvest 1, 8 days; Harvest 2, 7 days; Harvest 3, 7 days. The roller bottle scale up portion of the process was run at a set point of 37.0° C. The roller bottle inoculation and production portions of the process were run at a set point of 36.0° C.

UVC treatment was completed using a helical flow UVC reactor (at 254 nm), with an irradiation intensity of 60 W/m2, with flow rates between 5 and 20 liters per hour (LPH). The medium was treated with the correct UVC dosage for the desired condition.

### Example 1

TABLE 1
Experimental Conditions - Example 1

| | UVC Dose | UVC Treatment | |
|---|---|---|---|
| Condition | (mJ/cm²) | Stages | Comments |
| Control | N/A | N/A | |
| UVC 125 | 125 | Scale up, | UVC treatment of |
| | | inoculation, | 1X medium at all |
| | | production | stages |
| UVC [2.5X] 125 | 125 | Scale up, | UVC treatment of |
| | | inoculation, | 2.5X medium at all |
| | | production | stages |
| UVC 175 | 175 | Scale up, | UVC treatment of |
| | | inoculation, | 1X medium at all |
| | | production | stages |
| UVC 125 (2.5X, | 125 | Scale up, | UVC treatment of |
| 1X, 2.5X) | | inoculation, | 2.5X medium |
| | | production | during scale-up, 1X |
| | | | medium at |
| | | | inoculation, and |
| | | | 2.5X medium |
| | | | during production |
| UVC 125 (1X, | 125 | Scale up, | UVC treatment of |
| 1X, 2.5X) | | inoculation, | 1X medium during |
| | | production | scale-up, 1X |
| | | | medium at |
| | | | inoculation, and |
| | | | 2.5X medium |
| | | | during production |

Several other conditions were added to assess specific process needs and to test the robustness of the process to UVC medium treatment. The manufacturing facility prepares Scale up Media and Enriched Production Medium in a concentrated state and performs in-line filtration during transfer into the supply tank. The medium is brought to its final 1× volume using a WFI flush through the in-line filters. Various combinations of concentrated and 1×UVC treated medium were tested in this experiment.

### Results

There were no differences between the UVC treated condition and control for cell growth or population doubling at any stage of the scale up process. Metabolic data of the UVC conditions were similar to control throughout all harvests of the production. Cellular attachment at Harvest 3 was similar to control for all of the UVC conditions. The 2.5×UVC 125 mJ/cm² condition demonstrated slightly decreased attachment compared to the other conditions, but it is anticipated that this would have presented no issues if observed in full scale production.

All UVC media treated conditions demonstrated a titer reduction. The 175 mJ/cm² condition demonstrated a consistently lower titer across harvests. Media treated while concentrated appeared to have a greater effect on titer at harvest 3, but more studies would be needed to confirm this.

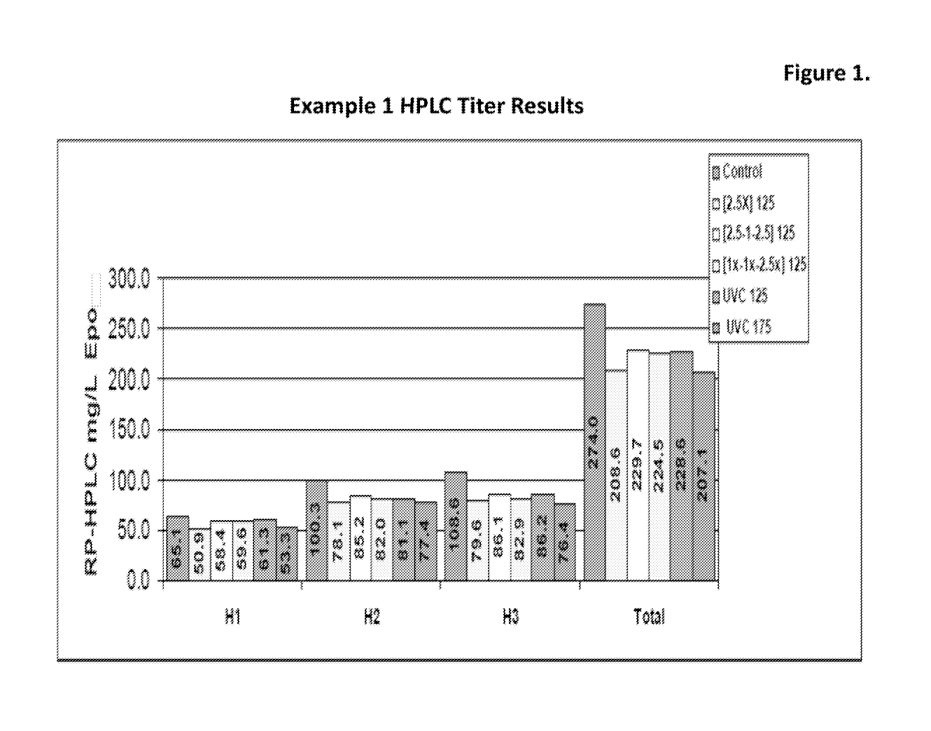

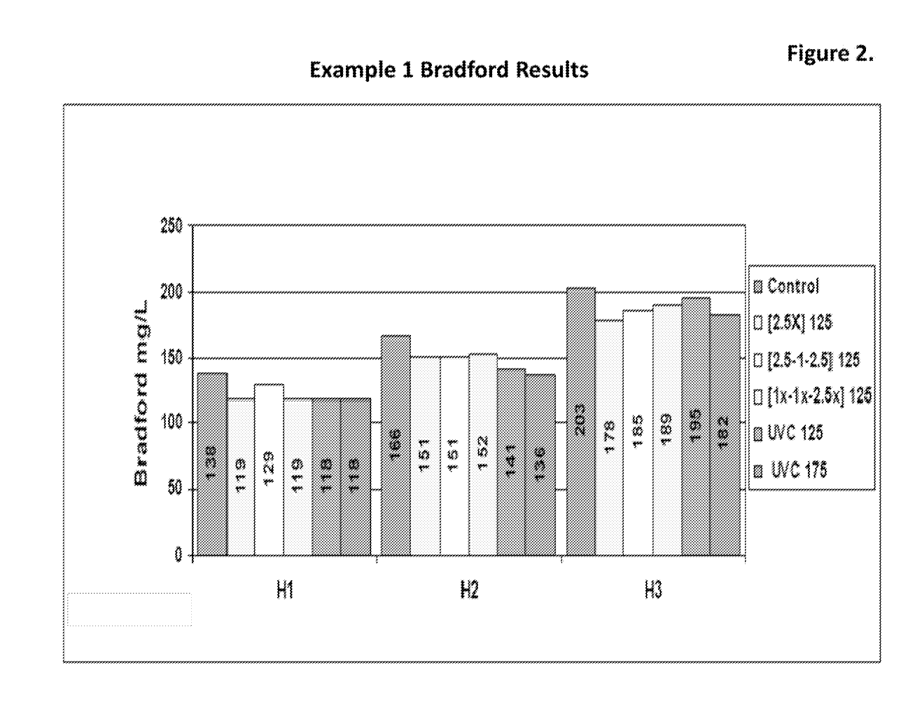

Only control and a repeat of the 2.5× concentrated scale up media that had been UVC treated was processed from vial thaw. The other UVC conditions started treatment at inoculation, continuing through production. Treatment starting at this point is representative of treatment from vial thaw. Product titer data is summarized in FIGS. 1 and 2.

### Example 2

TABLE 2
Experimental Conditions - Example 2

| | UVC Dose | UVC Treatment | |
|---|---|---|---|
| Condition | (mJ/cm²) | Stages | Comments |
| Control | N/A | N/A | |
| UVC 2.5X 125 | 125 | Scale up | Medium UVC |
| | | | treated at 2.5X |
| | | | concentration only |
| | | | in scale-up for |
| | | | growth comparison |
| UVC 50 [1X] | 50 | Inoculation, | |
| | | production | |
| UVC 75 [1X] | 75 | Inoculation, | |
| | | production | |

This experiment was run to repeat the scale up portion of the process with 2.5× concentrated UVC treated media and to assess lower UVC dosages during inoculation and production portions of the process. Previous experiments suggested that product titer is correlated to UVC dose, so lower dosages of 75 mJ/cm² and 50 mJ/cm² were tested.

### Results

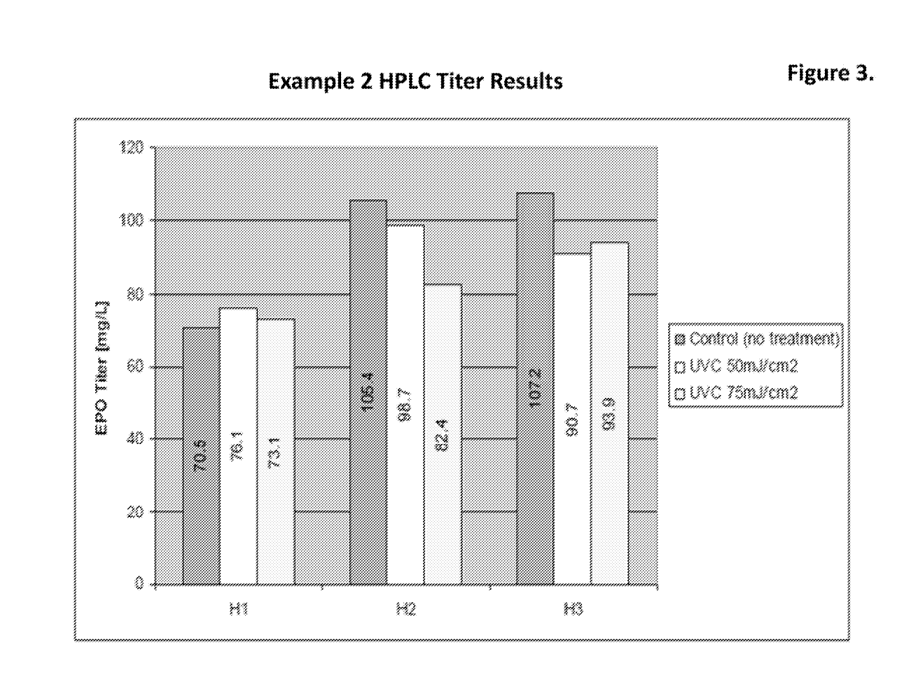

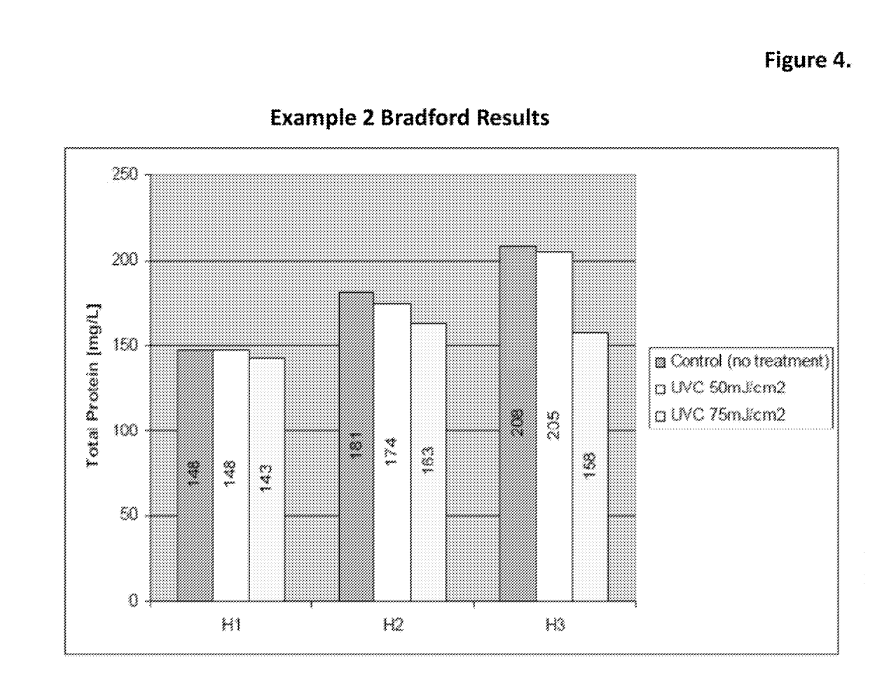

Metabolic data at shift and at each harvest of the production for all UVC treated conditions were comparable to control. Product titer demonstrated a dose dependent decrease with the two UVC treatments. Product titer data is summarized in FIGS. 3 and 4.

### Example 3

Various cell culture media treatment conditions were studied in this Epoetin alfa media treatment experiment. Media treatment technologies studied included high-temperature short-time (HTST), ultraviolet light-C (UVC) and viral filtration (VF). The treatments were applied starting at the roller bottle (RB) inoculation through production. Each condition was harvested and purified. All new UVC treatment conditions remediated the decreased titer observed with UVC treatment of current media (condition 2). All treated conditions also exhibited similar cell growth and viability at shift when compared to the control conditions (conditions 1 and 10). Some product quality differences were observed, such as lower SE-HPLC % monomer relative to the controls.

### Media Treatment Conditions

Table 3 shows the media treatment conditions applied to the various solutions used during production. Merged cells under the treatment columns of the table indicate that the treatment technology is applied to both components formulated together; separate cells under the treatment columns of the table indicate that the treatment technology is applied to each component separately.

TABLE 3
Summary of experimental conditions evaluated

Treatment(s)

| | | |
|---|---|---|
| Condition | Current DMEM | FBS |

| | |
|---|---|
| 1 and 10 | Control - no treatment |
| 2 | UVC (Bayer) |

| | | |
|---|---|---|
| 3 | HTST | UVC (Bayer) - 4x |
| | | diluted FBS |
| 4 | VF | UVC (Bayer) - 4x |
| | | diluted FBS |

| | | | |
|---|---|---|---|
| Condition | DMEM_S | DMEM_B | FBS |

| | |
|---|---|
| 5 | Control - no treatment |

| | | |
|---|---|---|
| 6 | VF | UVC (Bayer) |
| 7 | VF | UVC (Atlantic) |
| 8 | HTST | UVC (Bayer) |
| 9 | HTST | UVC (Atlantic) |

DMEM = current media
DMEM_S = additive package with UVC sensitive components
DMEM_B = new base media without UVC sensitive components
UVC = ultraviolet light-C
HTST = high temperature short time
VF = viral filtration
FBS = fetal bovine serum

Conditions 6-9 use the new base media that was UVC treated, followed by addition of the additive package, comprising lipoic acid, histidine, phenylalanine, tryptophan, tyrosine, folic acid, niacinamide, pyridoxal, pyridoxine, riboflavin, thiamine, methotrexate, and vitamin B12.

The target mean UVC dose for each condition of 125 mJ/cm² was not achieved precisely. This was due to variability in the fluorescent intensities in the original mean dose model. A more precise dose distribution model has subsequently been developed and utilized to report the mean and percentage (10, 50 and 90) doses. Mean doses delivered by the helical UVC reactor unit were lower than the target. The under-dosing of current media (condition 2) still resulted in decreased titers, which is consistent with prior studies that showed titer impacts for doses as low as 50 to 75 mJ/cm².

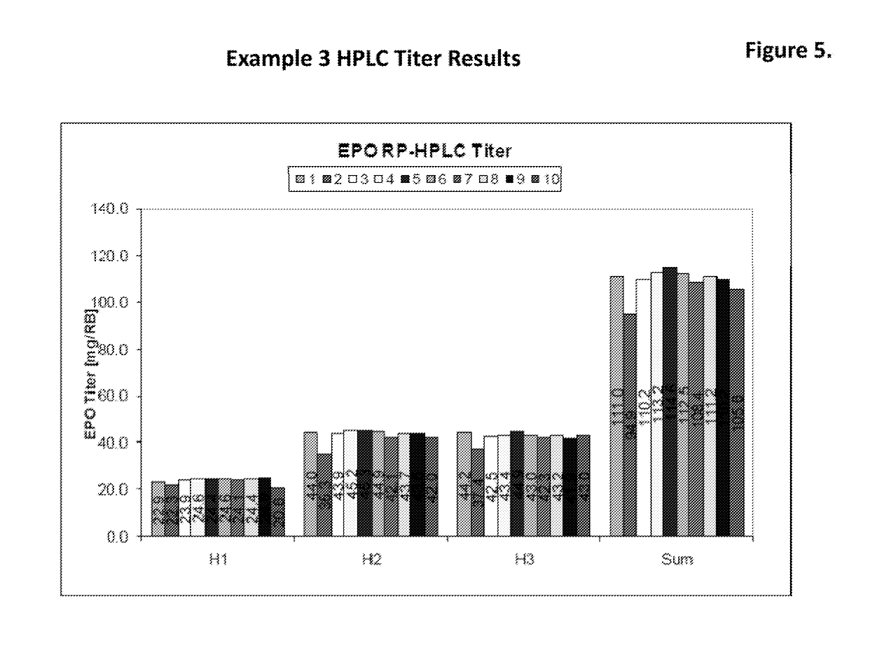

Productivity results are provided in FIG. 5. Treatment conditions 3, 4 and 6 through 9 had similar productivities within the action limits, thus remediating the titer decrease observed with UVC treatment of current media (condition 2), which was approximately 15-20% lower than all other conditions for harvests 2 and 3, and is consistent with prior studies. Results for the new media control, condition 5, were consistent with the existing media controls 1 and 10.

What is claimed is:

1. A cell culture media comprising:
a. a base media that is exposed to UVC light; and
b. an additive package comprising UV sensitive media components that is added to said base media after UVC exposure

2. The cell culture media of claim 1, wherein the base media does not comprise at least one, two, three, four, five, six, seven, eight, nine, ten, eleven, twelve, or thirteen components selected from the group consisting of lipoic acid, histidine, phenylalanine, tryptophan, tyrosine, folic acid, niacinamide, pyridoxal, pyridoxine, riboflavin, thiamine, methotrexate, and vitamin B12.

3. The cell culture media of claim 1, wherein the additive package comprises at least one, two, three, four, five, six, seven, eight, nine, ten, eleven, twelve, or thirteen components selected from the group consisting of lipoic acid, histidine, phenylalanine, tryptophan, tyrosine, folic acid, niacinamide, pyridoxal, pyridoxine, riboflavin, thiamine, methotrexate, and vitamin B12.

4. The cell culture media of any of claims 1-3, wherein the base media is in a powder or liquid form, and said additive package is in a powder or liquid form.

5. The cell culture media of claim 1, wherein the media is suitable for culture of mammalian cells.

6. The cell culture media of claim 1, wherein the media is suitable for culture of insect cells.

7. A method for making a UVC exposed cell culture media formulation, the method comprising the steps of
a. exposing a base media to UVC light;
b. adding an additive package comprising UV sensitive components to said UVC exposed base media.

8. The method of claim 7, wherein the base media does not comprise at least one, two, three, four, five, six, seven, eight, nine, ten, eleven, twelve, or thirteen components selected from the group consisting of lipoic acid, histidine, phenylalanine, tryptophan, tyrosine, folic acid, niacinamide, pyridoxal, pyridoxine, riboflavin, thiamine, methotrexate, and vitamin B12.

9. The method of claim 7, wherein the additive package comprises at least one, two, three, four, five, six, seven, eight, nine, ten, eleven, twelve, or thirteen components selected from the group consisting of lipoic acid, histidine, phenylalanine, tryptophan, tyrosine, folic acid, niacinamide, pyridoxal, pyridoxine, riboflavin, thiamine, methotrexate, and vitamin B12.

10. The method of any of claims 7-9, wherein the UVC light is at a wavelength of about 254 nm.

11. The method of claim 10, wherein the base media is exposed to UVC light at an energy density of about 25 to about 350 mJ/cm².

12. The method of claim 11, wherein the base media is exposed to UVC light at an energy density of about 125 mJ/cm².

13. The method of claim 11, wherein the base media is exposed to UVC light at an energy density of about 175 mJ/cm².

14. The method of claim 7, wherein the step of exposing the base media to UVC light is sufficient to damage the nucleic acids of any non-enveloped viruses in the base media.

15. The method of claim 7, wherein the UVC light is delivered using a thin film UVC reactor.

16. The method of claim 7, wherein the UVC light is delivered using a helical UVC reactor.

17. A method for producing a protein, the method comprising the steps of
a. exposing a base media to UVC light;
b. adding an additive package comprising UV sensitive media components to said UVC exposed base media; and
c. culturing cells in the UVC treated media such that a desired protein is produced.

18. The method of claim 17, wherein the base media does not comprise at least one, two, three, four, five, six, seven, eight, nine, ten, eleven, twelve, or thirteen components selected from the group consisting of lipoic acid, histidine, phenylalanine, tryptophan, tyrosine, folic acid, niacinamide, pyridoxal, pyridoxine, riboflavin, thiamine, methotrexate, and vitamin B12.

19. The method of claim 17, wherein the package comprises at least one, two, three, four, five, six, seven, eight, nine, ten, eleven, twelve, or thirteen components selected from the group consisting of lipoic acid, histidine, phenylalanine, tryptophan, tyrosine, folic acid, niacinamide, pyridoxal, pyridoxine, riboflavin, thiamine, methotrexate, and vitamin B12.

20. The method of any of claims 17-19, wherein the UVC light is at a wavelength of about 254 nm.

21. The method of claim 20, wherein the media is exposed to UVC light at an energy density of about 25 to about 350 mJ/cm².

22. The method of claim 21, wherein the base media is exposed to UVC light at an energy density of about 125 mJ/cm².

23. The method of claim 21, wherein the base media is exposed to UVC light at an energy density of about 175 mJ/cm².

24. The method of claim 17, wherein the step of exposing the base media to UVC light is sufficient to damage the nucleic acids of any non-enveloped viruses in the base media.

25. The method of claim 17, wherein the cells are CHO cells.

26. The method of claim 17, wherein the protein is recombinant human erythropoietin.

27. The method of claim 17, wherein the UVC light is delivered using a thin film UVC reactor.

28. The method of claim 17, wherein the UVC light is delivered using a helical UVC reactor.
